(19) United States
(12) Patent Application Publication
(10) Pub. No.: US 2026/0297164 A1
(43) Pub. Date: Oct. 1, 2026

(54) ANTI-FIBROTIC PEPTIDES AND USES THEREOF

(71) Applicant: Deackt, Inc., Houston, TX (US)

(72) Inventors: Ivan O. ROSAS, Houston, TX (US); James WHITEFORD, Houston, TX (US); Yuanyuan SHI, Houston, TX (US)

(21) Appl. No.: 19/159,108

(22) PCT Filed: Feb. 23, 2022
(86) PCT No.: PCT/CN2022/077425

§ 371 Date: Aug. 22, 2025

Related U.S. Application Data
(60) Provisional application No. 63/153,893, filed on Feb. 25, 2021.

(51) Int. Cl. C07K 14/705; A61K 38/00; A61P 11/00

(52) U.S. Cl. CPC C07K 14/70596; A61P 11/00; A61K 38/00

(57) ABSTRACT

Disclosed is an anti-fibrotic peptide, a source of the anti-fibrotic peptide, a pharmaceutical composition comprising the anti-fibrotic peptide and a method of treating fibrosis, a fibrotic condition or a fibrotic symptom using the anti-fibrotic peptide.

### CROSS REFERENCE TO RELATED APPLICATIONS

This application claims the priority of U.S. Provisional Patent Application No. 63/153,893, filed on Feb. 25, 2021, the disclosure of which is hereby incorporated by reference.

### FIELD

The present disclosure relates to the field of biological technology and pharmaceuticals, and particularly to anti-fibrotic peptides and uses thereof.

### BACKGROUND

The receptor-like protein tyrosine phosphatase-eta (PTPRJ/CD148) is expressed throughout the hematopoietic system and in the lung, pancreas, thyroid, kidney, mammary glands and nervous system. CD148 consists of 1,337 amino acids with a single phosphatase domain containing a conserved motif common to protein tyrosine phosphatases (PTPs). CD148 can regulate cell proliferation, apoptosis, migration and invasion in multiple cancers. CD148 can dephosphorylate and inactivate proteins that regulate mitogenic signals (i.e., PDGF, EGF and VEGF) and act as a putative negative regulator of growth factor receptor signaling, via PTP activity. Moreover, CD148 can negatively regulate PI3K/Akt signaling by dephosphorylating p85 (the regulatory subunit of PI3K).

Syndecans are a family of transmembrane receptors with roles in cell adhesion, migration and growth factor signalling. Each syndecan molecule comprises a short highly conserved cytoplasmic domain, a transmembrane domain and a larger extracellular domain (ectodomain). In mammals, there are four syndecan family members, syndecans-1, -2, -3 and -4. In common with the other family members syndecan-2 has a short cytoplasmic domain, a single pass transmembrane domain and a larger extracellular domain which is substituted toward the N-terminus with heparan sulphate (HS) side chains and can be shed from the cell surface. Syndecan shedding is a feature of many cell types and occurs in response to stimuli such as inflammatory mediators and growth factors. Syndecan-2 and CD148 are molecules intimately associated with the vasculature.

QM107 is an 18aa peptide (Sequence: PAEEDTNVYTEKHSDSLF, SEQ ID NO: 1) derived from human form of the proteoglycan cell adhesion receptor Syndecan-2 (SDC2). We have previously patented (Patent number: WO 2016/063042 A1, the disclosure of which is hereby incorporated by reference herein) this molecule for use as an inhibitor of angiogenesis in diseases where this process occurs. The inventors of the present disclosure have surprisingly found that a portion of the syndecan-2 molecule has an unexpected anti-fibrotic effect.

### SUMMARY

The present disclosure provides peptides with anti-fibrotic activity and nucleic acids that encode these peptides. The invention also provides methods of inhibiting αSMA expression in cells, method of inhibiting matrix production in cells, methods of inhibiting pro-fibrotic protein expression in cells, method of inhibiting collagen deposition and method of inhibiting structural damage in the lung. Further, the present disclosure provides methods, pharmaceutical compositions and kits for treating fibrosis, a fibrotic condition or a fibrotic symptom in a subject in need thereof.

In a first aspect, the present disclosure provides an anti-fibrotic peptide comprising or consisting of an amino acid sequence having at least 66%, 70%, 72%, 75%, 77%, 80%, 83%, 85%, 88%, 90%, 94%, 95% or 100% identity to the sequence of QM107 or mQM107.

QM107: anti-fibrotic peptide of 18 amino acid sequence (PAEEDTNVYTEKHSDSLF, SEQ ID NO: 1), which is derived from human syndecan-2 at positions 123-140.

mQM107: anti-fibrotic peptide of 18 amino acid sequence (PAIKSTDVYTEKHSDNLF, SEQ ID NO: 2), which is derived from mouse syndecan-2 at positions 124-141.

A “peptide” refers to a chain of amino acid residues linked by peptide bonds. The terms “peptide” and “polypeptide” are used interchangeably.

Throughout this specification, amino acids may be referred to using the three letter and one letter codes as follows: glycine (G or Gly), alanine (A or Ala), valine (V or Val), leucine (L or Leu), isoleucine (I or Ile), proline (P or Pro), phenylalanine (F or Phe), tyrosine (Y or Tyr), tryptophan (W or Trp), lysine (K or Lys), arginine (R or Arg), histidine (H or His), aspartic acid (D or Asp), glutamic acid (E or Glu), asparagine (N or Asn), glutamine (Q or Gln), cysteine (C or Cys), methionine (M or Met), serine (S or Ser) and Threonine (T or Thr). Where a residue may be aspartic acid or asparagine, the symbols Asx or B may be used. Where a residue may be glutamic acid or glutamine, the symbols Glx or Z may be used. References to aspartic acid include aspartate, and references to glutamic acid include glutamate, unless the context specifies otherwise.

In an embodiment of the first aspect, the peptide consists of up to 20, 25, 30, 40, 50, 60, 70, 80, 90 or 100 amino acids and includes an amino acid sequence having at least 66%, 70%, 72%, 75%, 77%, 80%, 83%, 85%, 88%, 90%, 94%, 95% or 100% identity to the sequence of QM107 or mQM107.

“Identity” as known in the art is the relationship between two or more polypeptide sequences or two or more polynucleotide sequences, as determined by comparing the sequences. In the art, identity also means the degree of sequence relatedness (homology) between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984)), BLASTP, BLASTN, and FASTA (Atschul et al., J. Molec. Biol. 215, 403 (1990)).

Peptides for use in the invention may be identical to one or more of the amino acid sequences disclosed herein apart from the substitution of one or more amino acids with one or more other amino acids. The skilled person is aware that various amino acids have similar properties. One or more such amino acids of a protein, polypeptide or peptide can often be substituted by one or more other such amino acids without eliminating a desired activity of that protein, polypeptide or peptide.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains).

Substitutions of this nature are often referred to as “conservative” or “semi-conservative” amino acid substitutions. The present invention therefore extends to use of a peptide comprising an amino acid sequence described above but with one or more conservative substitutions in the sequence, such that the amino acid sequence has at least 70% identity to those described herein.

The term “comprise” as used herein means that the claim encompasses all the listed elements or method steps, but may also include additional, unnamed elements or method steps. The peptide may comprise or consist of any of the amino acid sequences disclosed herein.

In a preferred embodiment of the first aspect, the peptide comprises or consists of an amino acid sequence having at least 66%, 70%, 72%, 75%, 77%, 80%, 83%, 85%, 88%, 90%, 94%, 95% or 100% identity to up to 20, 25, 30, 40, 50, 60, 70, 80, 90 or 100 consecutive amino acid residues derived from human syndecan-2 or mouse syndecan-2.

Human syndecan-2:
(SEQ ID NO: 3)
MRRAWILLTLGLVACVSAESRAELTSDKDMYLDNSSIEEASGVY
PIDDDDYASASGSGADEDVESPELTTSRPLPKILLTSAAPKVETT
TLNIQNKIPAQTKSPEETDKEKVHLSDSERKMDPAEEDTNVYTEK
HSDSLFKRTEVLAAVIAGGVIGFLFAIFLILLLVYRMRKKDEGSY
DLGERKPSSAAYQKAPTKEFYA.
QM107 corresponds to P123-F140 (underlined)
of the human Syndecan-2 sequence.
Mouse syndecan-2:
(SEQ ID NO: 4)
MQRAWILLTLGLMACVSAETRTELTSDKDMYLDNSSIEEASGVYP
IDDDDYSSASGSGADEDIESPVLTTSQLIPRIPLTSAASPKVETM
TLKTQSITPAQTESPEETDKEEVDISEAEEKLGPAIKSTDVYTEK
HSDNLFKRTEVLAAVIAGGVIGFLFAIFLILLLVYRMRKKDEGSY
DLGERKPSSAAYQKAPTKEFYA.
mQM107 corresponds to P124-F141
(underlined) of the mouse Syndecan-2 sequence.

In another embodiment of the first aspect, the peptide consists of up to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180 or 190 amino acid residues.

Peptides of the invention may be 15, 16, 17, 18, 19, 20, 21, 22, 23, 24 or 25 amino acid residues in length. In a preferred embodiment, the anti-fibrotic peptide consists of up to 25 amino acids comprising a) amino acid residues 123-140 of SEQ ID NO: 3 or amino acid residues 124-141 of SEQ ID NO: 4, or b) amino acid residues 123-140 of SEQ ID NO: 3 and having conservative amino acid substitutions at one or more positions selected from the group consisting of E125, E126, D127, N129, and S138, or amino acid residues 124-141 of SEQ ID NO: 4 and having conservative amino acid substitutions at one or more positions selected from the group consisting of I126, K127, S128, D130, and N139. In another preferred embodiment, the peptide consists of 18 or 19 amino acids and includes a) amino acid residues 123-140 of SEQ ID NO: 3 or amino acid residues 124-141 of SEQ ID NO: 4, or b) amino acid residues 123-140 of SEQ ID NO: 3 and having conservative amino acid substitutions at one or more positions selected from the group consisting of E125, E126, D127, N129, and S138, or amino acid residues 124-141 of SEQ ID NO: 4 and having conservative amino acid substitutions at one or more positions selected from the group consisting of 1126, K127, S128, D130, and N139.

In another embodiment of the first aspect, the peptide is fused to a heterologous peptide.

A “heterologous peptide” as used herein refers to a peptide that imparts desired characteristics to the anti-angiogenic peptide for example increased stability, enhanced transport or simplified purification or detection. The heterologous peptide is typically not a syndecan or derived from a syndecan.

Examples of heterologous peptides intended for the purposes of the present invention include glutathione S-transferase, polyhistidine or myc tag to facilitate purification of the polypeptide for example by affinity chromatography. In another embodiment, the heterologous peptide is a fluorescent polypeptide. Fluorescent polypeptides include but are not limited to green fluorescent protein, red fluorescent protein, yellow fluorescent protein, cyan fluorescent protein and their derivatives. In another embodiment, the heterologous peptide is intended to extend half-life of the fusion in circulation, Examples of the heterologous peptide include but does not limited to Fc fragment, serum albumin, Fc-binding peptide, and albumin-binding peptide.

Peptides of the invention may be produced by recombinant means, for example by expression of a nucleic acid construct as disclosed herein in a suitable vector, or by solid phase synthesis.

It should be appreciated that amino acid substitutions or insertions to the sequences disclosed herein that are within the scope of the present invention can be made using naturally occurring or non-naturally occurring amino acids. For example, D-amino acids can be incorporated in the peptides of the invention.

Peptides of the invention may be modified to improve their characteristics such as their half-life, for example by PEGylation.

In another embodiment of the first aspect, the peptide is conjugated to a carrier molecule.

In a second aspect, the present disclosure provides a source of the anti-fibrotic peptide of the first aspect. The term “source of the anti-fibrotic peptide” refers to a substance or composition which produces, expresses or releases the anti-fibrotic peptides of the present disclosure.

In an embodiment of the second aspect, the source is a polynucleotide which expresses the anti-fibrotic peptide. In another embodiment, the source is a polypeptide which is enzymatically cleavable to produce the anti-fibrotic peptide. In another embodiment, the source is a cell which expresses the anti-fibrotic peptide. In another embodiment, the source is a composition which releases the anti-fibrotic peptide.

In a third aspect, the present disclosure provides a pharmaceutical composition comprising the anti-fibrotic peptide of the first aspect or the source of the second aspect, and a pharmaceutically acceptable excipient or carrier.

A pharmaceutical composition according to the present invention may be presented in a form that is ready for immediate use. Alternatively, the composition may be presented in a form that requires some preparation prior to administration.

Pharmaceutical compositions of the invention may be adapted for administration by any appropriate route, for example by the oral (including buccal or sublingual), topical (including buccal, sublingual or transdermal), or parenteral (including subcutaneous, intramuscular, intravenous, intraperitoneal or intradermal) route.

The pharmaceutically acceptable carrier that is present in the pharmaceutical compositions of the invention may be any suitable pharmaceutically acceptable carrier or excipient that is known in the art.

Pharmaceutical compositions adapted for parenteral administration may include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents.

Excipients which may be used for injectable solutions include water, alcohols, polyols, glycerine and vegetable oils, for example. The compositions may be presented in unit-dose or multidose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

The pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants. They may also contain therapeutically active agents in addition to the peptide or nucleic acid construct of the present invention.

In a fourth aspect, the present disclosure provides a method of inhibiting αSMA expression in cells, comprising exposing the cells to the anti-fibrotic peptide of the first aspect, the source of the second aspect, or the pharmaceutical composition of the third aspect.

In an embodiment of the fourth aspect, the αSMA expression is induced by TGF-β1. In a preferred embodiment, the cells are lung cells, particularly fibroblasts.

In a fifth aspect, the present disclosure provides a method of inhibiting matrix production in cells, comprising exposing the cells to the anti-fibrotic peptide of the first aspect, the source of the second aspect, or the pharmaceutical composition of the third aspect.

In an embodiment of the fifth aspect, the αSMA expression is induced by TGF-β1. In a preferred embodiment, the cells are lung cells, particularly fibroblasts.

In a sixth aspect, the present disclosure provides a method of inhibiting pro-fibrotic protein expression in cells, comprising exposing the cells to the anti-fibrotic peptide of the first aspect, the source of the second aspect, or the pharmaceutical composition of the third aspect, preferably the pro-fibrotic protein includes at least one or two selected from the group consisting of collagen 1a1 and fibronectin.

In an embodiment of the sixth aspect, the αSMA expression is induced by TGF-β1. In a preferred embodiment, the cells are lung cells, particularly fibroblasts.

In a seventh aspect, the present disclosure provides a method of treating fibrosis, a fibrotic condition or a fibrotic symptom in a subject in need thereof, comprising administering to the subject a therapeutically effective amount of the anti-fibrotic peptide of the first aspect, the source of the second aspect, or the pharmaceutical composition of the third aspect.

A therapeutically effective amount is the dose sufficient to reduce or inhibit fibrosis.

Doses for delivery and administration can be based upon current existing protocols, empirically determined, using animal disease models or optionally in human clinical trials. Initial study doses can be based upon animal studies set forth herein, for a mouse, for example.

Doses can vary and depend upon whether the treatment is prophylactic or therapeutic, the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled person will appreciate the factors that may influence the dosage and timing required to provide an amount sufficient for providing a therapeutic or prophylactic benefit.

As used herein, a subject refers to an animal, including a human being. An animal can include mice, rats, fowls such as chicken, ruminants such as cows, goat, deer, sheep and other animals such as pigs, cats, dogs and primates such as humans, chimpanzees, gorillas and monkeys. Preferably the subject is human.

In an embodiment of the seventh aspect, the fibrosis, fibrotic condition or fibrotic symptom is induced by TGF-β1, bleomycin or by radiation.

In a preferred embodiment, the subject has pulmonary fibrosis.

In another preferred embodiment, the administration is topical administration, particularly intranasal administration.

In another preferred embodiment, the anti-fibrotic peptide, the source or the pharmaceutical composition is in a spray composition or inhalation composition.

In an eighth aspect, the present disclosure provides a method of inhibiting collagen deposition in the lung of a subject in need thereof, comprising administering to the subject a therapeutically effective amount of the anti-fibrotic peptide of the first aspect, the source of the second aspect, or the pharmaceutical composition of the third aspect.

In a ninth aspect, the present disclosure provides a method of inhibiting structural damage in the lung of a subject in need thereof, comprising administering to the subject a therapeutically effective amount of the anti-fibrotic peptide of the first aspect, the source of the second aspect, or the pharmaceutical composition of the third aspect.

In an embodiment of the seventh, eighth or ninth aspect, the subject has a fibrotic condition selected from the group consisting of glycogen storage disease type III (GSD III), glycogen storage disease type VI (GSD VI), glycogen storage disease type IX (GSD IX), nonalcoholic steatohepatitis (NASH), cirrhosis, hepatitis, scleroderma, alcoholic fatty liver disease, atherosclerosis, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, pulmonary fibrosis, idiopathic pulmonary fibrosis, diffuse parenchymal lung disease, idiopathic interstitial fibrosis, diffuse interstitial fibrosis, interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-poly myositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis, or symptoms or sequelae thereof, or other diseases or conditions resulting in the excessive deposition of extracellular matrix components, such as collagen, which may be affected by interventions within the TRJ3 pathway, dust lung, sarcoidosis, lung fibrosis induced by drug or radiation fibrogenic alveolitis, and a combination thereof.

In another embodiment, the subject is suffering a disease selected from the group consisting of idiopathic pulmonary fibrosis (IPF), hypersensitivity pneumonitis, dust lung, sarcoidosis, lung fibrosis induced by drug or radiation and fibrogenic alveolitis.

In a tenth aspect, the present disclosure provides a method of treating a cancer in a subject in need thereof, comprising administering radiation therapy in combination with a therapeutically effective amount of the anti-fibrotic peptide of the first aspect, the source of the second aspect, or the pharmaceutical composition of the third aspect.

In another aspect, the present disclosure provides a method of treating a cancer in a subject receives radiation therapy, comprising administering radiation therapy in combination with a therapeutically effective amount of the anti-fibrotic peptide of the first aspect, the source of the second aspect, or the pharmaceutical composition of the third aspect.

The present disclosure also provides the anti-fibrotic peptide of the first aspect, the source of the second aspect, or the pharmaceutical composition of the third aspect for use in the methods of any of the fourth aspect to the tenth aspect.

The present disclosure also provides the use of the anti-fibrotic peptide of the first aspect, the source of the second aspect, or the pharmaceutical composition of the third aspect for the manufacture of a medicament for the method of the fourth aspect to the tenth aspect.

### BRIEF DESCRIPTION OF DRAWINGS

FIG. 1. CD148 is downregulated in IPF lung and contributes to pro-fibrotic phenotype of IPF-derived lung fibroblasts. (A) CD148 protein expression levels were determined in lung homogenates from control (n=5) and IPF (n=5) patients. (Graphic) Densitometry (by ImageJ software). (B) Lung specimens from control (n=9) and IPF (n=10) patients were homogenized and subjected to total RNA isolation. Ptprj (CD148) mRNA levels were assessed using qPCR. (C) Single cell suspensions of control (n=6) and IPF (n=7) were enriched for fibroblasts. mRNA expression of CD148 in fibroblast-enriched cell populations were assessed using qPCR. (D) Representative fluorescence microscopy images of CD148 (Cy3, red), Vimentin (GFP, green) and DAPI (blue) in control (n=4) and IPF lung tissue (n=5). Scale bar=100 μm. Enlarged areas (Clockwise from top right panel) represent Vimentin, CD148, DAPI and merged image. CD148 fluorescence intensity was quantified by ImageJ. (E-F) Lung fibroblasts from non-disease (control) and IPF samples were transfected with scramble (Scr) or shCD148. (E) CD148 protein levels were measured by western blot (n=3). (F) mRNA levels of fibronectin (Fn) and Collagen 1a1 (Col1a1) were measured using qPCR (n=5). (G) Scr and shCD148 transfected cells were seeded in 24 well plates and then treated with Fas ligand (FasL, 200 ng/ml) for 24 h. After treatment, cell viability was determined using the MTT assay (n=5). (H) IPF-derived lung fibroblasts were stably transfected with empty vector ((EV, pLenti-GIII-HA) or pLenti-GIII-CD148-HA (CD148-HA). The cells were lysed and subjected to western blot to measure hemagglutinin tag (HA) (n=3). (I) In EV and CD148-HA transfected cells, the expression of Fn and Col1a1 were measured using qPCR (n=5). (J) EV and CD148-HA transfected cells were seeded at equal amounts in 24 well plates and treated with FasL (200 ng/ml) for 24 h. Cell viability was determined using the MTT assay (n=5). Data are mean±s.e.m. *P<0.05, by Mann-Whitney's unpaired non-parametric test (E and H), Student's unpaired t test (B and C), one-way ANOVA (F, G, I, J). (K) CD148 gene expression in fibroblasts and myofibroblasts from single cell RNA sequencing (scRNA-seq). Clusters labeled as fibroblast and myofibroblast were extracted from our previously published scRNA-seq data set and analysed using R-program. Data are presented as box and whiskers plot. P values were calculated by Mann-Whitney unpaired test. (L) mRNA expression of CD148 in sorted AT1 and AT2 cells were assessed using qPCR. (M) Control and IPF derived lung fibroblasts were seeded at equal amount in 24 well plates and then treated with Fas ligand (FasL) (100 and 200 ng/ml) for 24 h. After treatment, cell viability was assessed with the MTT assay (n=4). (N) EV and CD148-HA transfected cells were seeded at equal amount in 24 well plates and treated with FasL (200 ng/ml) for 24 h. After treatment, cells were subjected to caspase-3 activity assay (n=5). Data are mean±s.e.m. *P<0.05, by Student's unpaired t test for L and one-way ANOVA for M, N.

FIG. 2. CD148 deficiency in fibroblasts worsens pulmonary fibrosis in bleomycin-treated mice and increases fibroblast activation in response to TGF-β1. CD148 fibroblast-specific knockout mice (Ptprj^{fl/fl} Col1a2^{Cre-ER(T)+/0} were developed as described in Methods. (A) Ptprj^{fl/fl} Col1a2^{Cre-ER(T)+/0} mice or Col1a2^{Cre-ER(T)+/0} (wild type) mice were exposed to BLM to induce lung fibrosis. At day 21, mouse lungs were harvested and stained with Masson's trichrome (n=3 for saline and n=5 for BLM groups). (B) Hydroxyproline content was measured in the left lung of Ptprj^{fl/fl} Col1a2^{Cre-ER(T)+/0} mice (n=12) and Col1a2^{Cre-ER(T)+/0} (WT) mice (n=12) exposed to BLM, and Ptprj^{fl/fl} Col1a2^{Cre-ER(T)+/0} (n=6) and Col1a2^{Cre-ER(T)+/0} (n=6) exposed to saline; at day 21. (C) Relative survival at day 0-21 after BLM. (D-E), α-SMA and CD148 expression in harvested lungs were measured by western blot (n=3-5 for each condition). (F-G) Gene expression of Fn and Col1a1, and Ctgf in harvested lungs was measured using qPCR (n=5 for each condition). (H-I), Mouse lung fibroblasts from Ptprj^{fl/fl} Col1a2^{Cre-ER(T)+/0} mice (Ptprj−/−) and Col1a2^{Cre-ER(T)+/0} (WT) mice were isolated and treated with 4-OHT (1 μM) for 24 h. After 4-OHT treatment, cells were exposed to TGF-β1 (10 ng/ml) for an additional 24 h. After stimulation, cells were harvested. (H) Western blot (n=4 for each condition). (I) mRNA levels of Col1a1 and Fn were measured by qPCR (n=5 for each condition). (J) Cells were mixed with collagen 1. Gel contractility was measured at 0, 12 and 24 h after TGF-β1 (10 ng/ml) stimulation (n=6 for each condition). (K) Cell death was induced by FasL (200 ng/ml). At 24 h, cell viability was measured with MTT assay (n=5 for each condition). Data are mean±s.e.m. *P<0.05, by one-way ANOVA. (L) WT and Ptprj−/− mouse lung fibroblasts were harvested and plated at 1×104 cells/well in the presence or absence of TGF-β1 (10 ng/ml). 3 days later, cells were harvested, and live cells were estimated using the trypan blue exclusion assay. Data are mean #s.e.m. *P<0.05.

FIG. 3. CD148 deficiency enhances PI3K/Akt/mTOR signaling which results in low autophagy, high p62 expression in lung fibroblasts. (A) Mouse lung fibroblasts were isolated from Ptprj^{fl/fl} Col1a2^{Cre-ER(T)+/0} mice (Ptprj−/−) and Col1a2^{Cre-ER(T)+/0} mice (WT) and then treated with 4-OHT (1 μM) for 24 h. After 4-OHT treatment, cells were exposed to TGF-β1 (10 ng/ml) for 24 h. After stimulation, cells were harvested, and the expression of p-p85 (Tyr458), p-Akt (Ser473), pmTOR (Ser2448), p-p70 S6 kinase (Thr389), p-S6 ribosomal protein (Ser235/236) and CD148 (n=4 for each condition) in lysates was determined by Western immunoblotting and corresponding densitometry (ImageJ software). Data were normalized to corresponding dephospho-forms or GAPDH. (B) WT and Ptprj−/− cells were stimulated with TGF-β1 (10 ng/ml) for 24 h. Then cells were lysed and LC3-I and -II and p62 were measured by western blot (n=4). (C) WT and Ptprj−/− cells were incubated in starvation media (Hank's buffered salt solution (HBSS, without calcium/magnesium) containing 1% regular medium) for 24 h. Then, autophagy flux was measured by LC3-II accumulation in the absence or presence of lysosomal acidification inhibitor chloroquine (25 μM) at 2, 4 and 6 h (n=4). (D-E) Lung fibroblasts from LC3-GFP transgenic mice were transfected with scr or shCD148 (lentivirus). Cells were starved for 24 h in the presence or absence of TGF-β1 (10 ng/ml), then cells were treated with chloroquine (25 μM) for 4 h. After treatment cells were fixed and digital images (3 images per sample) were taken using fluorescent microscope. Representative images are shown in (E). LC3 puncta positive cells were quantified using ImageJ (D). Data are mean #s.e.m. *P<0.05, by one-way ANOVA. (F) Increase of ECM expression in CD148-deficient cells in response to TGF-β1 is dependent on the PI3K/mTOR axis and p62. Ptprj−/− cells were transfected with scr or shp62 (lentivirus). Cells were stimulated with TGF-β1 (10 ng/ml) in the presence or absence of wortmannin (wort, 50 nM) or rapamycin (rapa, 1 μM). 24 h cells were lysed, and subjected to western blot (α-SMA, n=3). GAPDH was the standard.

FIG. 4. CD148 deficiency enhances PI3K/Akt/mTOR signaling which results in enhanced NF-κB activation in lung fibroblasts. (A) WT and Ptprj−/− cells were stimulated with TGF-β1 (10 ng/ml) for 24 h. Cells were lysed and p-IKKα/β, p-IκB and IκB were measured by western blot (n=4). GAPDH was the standard. Bar graphs at right are the quantitation of corresponding proteins. (B) WT and Ptprj−/− cells were stimulated with TGF-β1 (10 ng/ml) for 24 h. Cells were subjected to cytosol and nuclear protein fractionation. The p65 (NF-κB subunit) nuclear translocation was measured by western blot (n=3). β-actin and PCNA were used as cytosolic and nuclear markers, respectively. Bar graphs at right are the quantitation of corresponding proteins. p65 cytosolic/nuclear ratio is shown (far right). Data are mean±s.e.m. *P<0.05, by Kurskal-Wallis non-parametric test (C-D) WT or Ptprj−/− cells were transfected with scr or shp62 (lentivirus). Cells were stimulated with TGF-β1 (10 ng/ml) in the presence or absence or wortmannin (wort, 50 nM) or rapamycin (rapa, 1 μM). At 24 h cells were harvested and subjected to qPCR for Col1a1 or Acta2 (n=5). (E) WT or Ptprj−/− cells were transfected with NF-κB luciferase reporter plasmid in the presence or absence of scr or shp62, or wort (50 nM) or rapa (1 μM). Then, cells were treated with TGF-β1 (10 ng/ml) for 4 h. Luciferase activity were measured as described in methods (n=4 or 7). Data are mean±s.e.m. *P<0.05, by one-way ANOVA. (F) WT or Ptprj−/− cells were stimulated with TGF-β1 (10 ng/ml) in the presence or absence of the NF-κB inhibitor Bay 11-7082 (10 μM). mRNA levels of Col1a1 were measured by qPCR (n=5 for each condition). Data are mean±s.e.m. *P<0.05, by one-way ANOVA. (G) CD148 overexpression inhibits NF-κB activation and p62 expression. Human lung fibroblasts (MRC-5 cells) were lentivirally transfected with empty vector (EV, pLenti-GIII-HA) or pLenti-GIII-CD148-HA (CD148-HA). Then cells were stimulated with TGF-β1 (10 ng/ml) for an additional 24 h. After stimulation, cells were harvested and lysates were subjected to western blot to measure p-IKKα/β, p-IκB and p62 (n=3).

FIG. 5. SDC2 18-aa peptide inhibits pulmonary fibrosis in vivo; upregulates autophagy by downregulation of PI3K/Akt/mTOR signaling and inhibits ECM gene expression in mouse fibroblasts via CD148. (A-C) Ptprj^{fl/fl} Col1a2^{Cre-ER(T)+/0} mice (fibroblast-specific CD148 deficient) and Col1a2^{Cre-ER(T)+/0} (WT) mice were exposed to BLM. SDC2 18-aa peptide (SDC2-pep, 0.5 mg/kg) was intranasally delivered into WT and Ptprj^{fl/fl} Col1a2^{Cre-ER(T)+/0} mice 10 days after BLM injury. Treatments were repeated at day 12, 14, 16 and 18. (A) At 24 days after BLM exposure lungs were harvested and stained with Masson's trichrome (n=3 for saline and n=5 for BLM groups). (B) Hydroxyproline content was measured in the left lung of mice exposed to BLM (n=11) or saline (n=5). Gene expression of (C) Fn and Col1a1; and (D) connective tissue growth factor (Ctgf) in harvested lungs were measured using qPCR (n=5 for each condition). (E-F) Mouse lung fibroblasts from Ptprj^{fl/fl} Col1a2^{Cre-ER(T)+/0} mice (Ptprj−/−) and Col1a2^{Cre-ER(T)+/0} mice (WT) were isolated and treated with 4-OHT (1 μM) for 24 h. After 4-OHT treatment, cells were exposed to ±TGF-β1 (10 ng/ml) for an additional 24 h, in the absence or presence of SDC2-pep (5 μM). (E) Expression of p-AKT/Akt, p-mTOR/mTOR, p62/GAPDH p-IKKα/β/IKKα, α-SMA/GAPDH, and CD148/GAPDH was determined by Western immunoblotting (n=4), and corresponding densitometry (ImageJ software). Data are mean±s.e.m. *P<0.05, by one-way ANOVA. (F) mRNA levels of Col1a1 and Fn were measured by qPCR (n=5 for each condition). (G) After treatment, cells were mixed with collagen 1. Gel contractility was measured at 0, 12, and 24 h after TGF-β1 stimulation (n=6 for each condition). Data are mean±s.e.m. *P<0.05, by one-way ANOVA. (H) SDC2-pep alleviates lung fibrosis after BLM-induced injury. Total cell concentrations were measured in BAL fluid from Ptprj^{fl/fl} Col1a2^{Cre-ER(T)+/0} mice and Col1a2-Cre (control) mice 21 days after BLM injury (n=5/group).

FIG. 6. SDC2 18-aa peptide attenuates pro-fibrotic gene expression in human IPF fibroblasts. (A) IPF-derived lung fibroblasts were transfected with scr or shCD148. Then cells were treated with SDC2 18-aa peptide (5 μM) for 24 h. After incubation, p-Akt (Ser473), p-mTOR (Ser2448), LC3, p62 and CD148 were measured by western blot (n=4). Band intensities were quantified using ImageJ software and were expressed as a ratio of band intensity relative to GAPDH. Data are mean±s.e.m. *P<0.05, by one-way ANOVA. (B) mRNA levels of Fn and Col1a1 were measured by qPCR (n=5). (C) Scr or shCD148 transfected control or IPF-derived lung fibroblasts were mixed with collagen 1. Gel contractility were measured at 0, 12 and 24 h in the presence or absence of SDC2-pep (5 μM), n=5/group. (D) Scr or shCD148 transfected control or IPF-derived lung fibroblasts were treated with FasL (200 ng/ml) for 24 h in the presence or absence of SDC2-pep (5 μM) (n=7). Cell viability was measured using MTT assay. Data are mean±s.e.m. *P<0.05, by one-way ANOVA.

FIG. 7. SDC2 18-aa peptide and CD148 overexpression attenuate pro-fibrotic gene expression in PCLSs derived from IPF lungs, and from wild type lungs subjected to profibrotic stimuli. (A-D) PCLS from IPF lungs were transfected with Scr, shCD148 or EV and CD148-HA. Then, PCLS were treated with SDC2-pep (5 μM) for 72 h. After incubation, PCLS were digested for total RNA isolation. The expression of (A) Fn and Col1a1, (B) Acta2, and (C) Ptprj (CD148) were measured by qPCR (n=7 for each condition). (D) mRNA levels of Col1a1, Fn, Acta2 and Ptprj (CD148) were measured by qPCR in EV and CD148 overexpressing PCLS (CD148-HA). (E-H) PCLS from control lungs were transfected with Scr, shCD148 (lentivirus). Then, PCLSs were treated with pro-fibrotic mix (FGF basic, 25 ng/ml, PDGF-BB, 10 ng/ml, TGF-β1, 10 ng/ml) 72 h with or without SDC2-pep (5 μM). After incubation, slices were digested for total RNA isolation. The expression of E, Fn, F, Col1a1, G, Acta2, and H, Ptprj were measured by qPCR. (A-H, n=6 for each condition). Data are mean±s.e.m. *P<0.05, by one-way ANOVA.

FIG. 8. Schematic overview of Tamoxifen treatments to induce conditional knockout of CD148 in fibroblasts.

FIG. 9. QM107 inhibits TGF-β1 induced αSMA production in Mouse Lung Fibroblasts.

FIG. 10. QM107 inhibits TGF-β1 stimulated matrix production in Mouse Lung Fibroblasts.

FIG. 11. Topical application of QM107 in the bleomycin lung fibrosis model leads to a reduction in collagen deposition.

FIG. 12. Histochemical staining of bleomycin treated lungs reveals QM107 leads to less collagen deposition and less structural damage.

FIG. 13. QM 107 attenuates pro-fibrotic gene expression in precision cut lung slices from IPF patients.

FIG. 14. Schema depicting proposed antifibrotic effects of CD148 in fibroblasts. Syndecan-2 (SDC2) via binding its receptor protein tyrosine phosphatase CD148/PTPRJ activates an anti-fibrotic pathway dependent on downregulation of TGF-β1-dependent signaling. TGF-β1 stimulates pro-fibrotic effects via its receptor TGFβ-I/-II complex which activate a PI3K/AKT/mTOR-dependent signaling pathway culminating in the suppression of autophagy. The autophagy pathway, driven by LC3-dependent formation of autophagosomes, directs the lysosomal degradation of autophagosome-sequestered cargo. Impaired autophagy is a characteristic feature of pulmonary fibrosis, which leads to aberrant accumulation of the autophagy substrate and cargo adaptor protein p62. Accumulated p62 promotes phosphorylation of the IKK complex, leading to phosphorylation and dissociation of I-κB from the p65 subunit of NF-κB. The latter promotes p65/p50 assembly and migration of the NF-κB complex to the nucleus, where it stimulates pro-fibrotic gene expression. NF-κB has also been implicated in apoptosis resistance and myofibroblast differentiation, characteristic of the pro-fibrotic phenotype. Sequences (upper right) depict an 18-aa peptide region of the SDC2 ectodomain (SDC2-pep) with a high degree of homology between human and mouse sequences. SDC2-pep was tested in the current study as a therapeutic ligand of CD148/PTPRJ and found to have anti-fibrotic effects in IPF and models of pulmonary fibrosis.

### DETAILED DESCRIPTION

The present disclosure will now be further described by way of reference to the following Examples which are present for the purposes of reference only and are not to be construed as being limiting on the present disclosure.

### EXAMPLES

### Methods and Materials

### Reagents

Antibodies against α-SMA (no. ab5694), collagen 1 (no. ab34710) and GAPDH (no. ab8245) were from Abcam. CD148/DEP1 (no. MAB1934) was from R&D systems. Hemagglutinin tag (HA-tag, no. G036) was from ABMgood (Richmond, BC, Canada). Antisera against phospho-Akt (Ser473, no. 9271), Akt (no. 9272), phospho-PI3K (p85 (Tyr458)/p55 (Tyr199), no. 4228), PI3K (p85, no. 4292), phospho-mTOR (Ser2448, no. 5536), mTOR (no. 2972), phospho-S6 ribosomal protein (Ser235/236, no. 2211), S6 ribosomal protein (no. 2217), phospho-p70 S6 kinase (Thr389, no. 9206), p70 S6 kinase (no. 2708), LC3a/b (no. 4108), p62 (no. 5114), phospho-IKKα/β (Ser176/180, no. 2697), IKKα (no. 2682), phospho-IκB-α (Ser32, no. 2859), I-κB-α (no. 4812), NF-κB (p65, no. 8242), β-actin (no. 4970), PCNA (no. 13110) were from Cell Signaling Technologies (Beverly, MA). All other reagents, including 4-hydroxy-tamoxifen, were from Millipore Sigma (St Louis, MO).

### Primary Pulmonary Fibroblasts

This study was approved by the institutional review board at Brigham and Women's Hospital (approval number: 2011P002419). Lung fibroblasts were derived from patients who were subjected to lung transplantation with progressing IPF. Primary control lung (control) fibroblasts were derived from nonfibrotic lung samples lacking any evidence of disease which were deemed unsuitable Mouse for transplantation. lung fibroblasts from Ptprj^{fl/fl}Col1a2-Cre-ER(T)^{+/0}, Col1a2-Cre-ER(T)^{+/0} or GFP-LC3 transgenic mice were obtained as previously described (Tsoyi K et al., Crit Care Med 2016; 44: e1236-e1245). Briefly, isolated lungs were minced and digested in collagenase buffer. Cell pellets will be resuspended and cultured for 7-10 days. Cells were lineage and Sca-1 depleted using commercially available kits from StemCell Technologies (Vancouver, BC) to remove hematopoietic and progenitor cell populations. Fibroblasts were cultured in complete media (DMEM; Corning) containing 10% FBS (Corning), 100 IU of penicillin and 100 μg/ml streptomycin (Corning), 292 μg/ml L-glutamine (Corning), and 100 μg/ml Primocin (InVivoGen) in humidified incubators at 37° C. and 10% CO₂. To induce Cre recombinase expression in Ptprj^{fl/fl}Col1a2-Cre-ER(T)^{+/0} or Col1a2-Cre-ER(T)^{+/0} lung fibroblasts, cells were treated with 4-hydroxytamoxifen (4-OHT, 1 μM).

### Mice

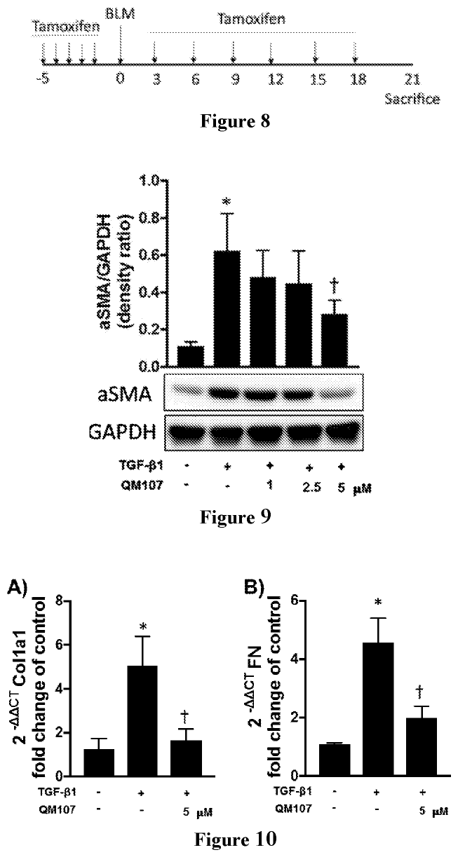

All animal experimental protocols were approved by the Brigham and Women's Hospital Standing Committee for Animal Welfare. WT C57BL/6 mice were obtained from Charles River Laboratories and used at 8 weeks of age. GFP-LC3 transgenic mice (Adolph T E et al., 2013; Nature 503:272-276) were kindly provided by Dr. Blumberg, Brigham and Women's Hospital, Boston, MA. Transgenic conditional PTPRJ/CD148-knockout mice of both sexes were bred as follows on a C57BL/6 background. Transgenic Col1a2^{Cre-ER(T)+/0} and Ptprj^{fl/fl} mice were obtained from Jackson Laboratory (no. 029567, no. 008291 respectively, Bar-Harbor, ME). To generate fibroblast-specific CD148-deficient mice, Ptprj“ ” mice were bred with Col1a2^{Cre-ER(T)+/0} (heterozygous allele) transgenic mice to generate mice heterozygous for both alleles. Progeny from the second cross between Ptprj^{fl/fl} mice and heterozygous Ptprj^{fl/WT} Col1a2^{Cre-ER(T)+/0} mice (from the first cross) were used for further experiments. All mice were genotyped by PCR techniques as described previously. For treatment of mice, a stock solution of tamoxifen (Sigma-Aldrich) was diluted in corn oil to 20 mg/ml. To selectively delete CD148 in activated fibroblasts, adult Ptprj^{fl/fl}Col1a2^{Cre-ER(T)+/0} mice (8-10 weeks old) and control Col1a2^{Cre-ER(T)+/0} mice were administered tamoxifen suspension (0.1 ml of diluted stock) via intraperitoneal (i.p.) injection (75 mg/kg), for 5 days before administration of BLM (0.75 mg/kg) and every 72 h thereafter until sacrifice as shown in FIG. 8.

### Bleomycin Model of Pulmonary Fibrosis

Lung fibrosis was elicited in mice by BLM (0.75 mg/kg, intratracheal) (Cayman Chemical); control mice received an equal volume of saline. Mice were sacrificed 21 days after BLM instillation. BAL fluids were analyzed for immune cell counts. The left lung was analyzed for hydroxyproline. The right lung lobes were assessed for gene expression (Acta2, Col1a1, Tgfb1, and Ctgf) and histology.

### Hydroxyproline Assay

To quantify collagen deposition, the left lung from each mouse was hydrolyzed in 6N HCl for 24 h at 110° C., and hydroxyproline levels were quantified. Each sample was tested in triplicate. Data are expressed as micrograms of hydroxyproline per left lung.

### SDC2-ED 18-Aa Peptide (SDC2-Pep) Administration

Ptprj^{fl/fl}Col1a2^{Cre-ER(T)+/0} (CD148 fibroblast-specific knockout) and Col1a2^{Cre-ER(T)+/0} (control) mice at 8 weeks of age were treated with tamoxifen and instilled with 0.75 mg/kg of BLM in 100 μl sterile saline at Day 0. Control animals were treated with an equal volume of sterile saline. In the treatment group, SDC2-ED 18-aa peptide (SDC2-pep, 0.5 mg/kg in 50 μl of phosphate-buffered saline [PBS]) was administered at days 10, 12, 14, 16 and 18 post-BLM or saline by oropharyngeal instillation. The control group was treated with an equal volume of sterile PBS (vehicle). Mice were sacrificed 24 days after BLM or saline treatment.

### Precision Cut Lung Slices (PCLS)

PCLS from control and IPF lungs were prepared as described (Uhl F E et al, Eur Respir J 2015; 46:1150-1166; Bai Y et al, Am J Respir Cell Mol Biol 2016; 54:656-663). Briefly, using a syringe pump, lungs were infiltrated with warm, 2% (37° C.) low-melting agarose-HBSS solution (Millipore Sigma, no. A9414; kept at 37° C.). After complete solidification of agarose in the inflated lobes on ice, tissue blocks of approximately 10 mm in diameter were prepared. Lung slices (300 μm thick) were cut perpendicularly to the visible airway with a vibratome (Precisionary Instruments, no. VF-300, Greenville, NC) at room temperature in HBSS. Then, slices were cultured in 24 well plates supplemented with DMEM/F12 media containing 1% FBS and antibiotics. Slices were transfected with scr, shCD148, EV or pLenti-GIII-CD148-HA lentiviral particles (1-1.5 MOI per slice). 12 h later, lung slices were incubated with or without SDC2-ED 18-aa peptide (5 μM) for another 72 h. After treatment, slices were subjected to total RNA isolation to measure profibrotic genes expression (Acta2, Col1a1 and Fn) or immunofluorescent staining.

### Histopathology and Immunofluorescent Co-Staining

Human lung sections were fixed by inflation with buffered 10% formalin solution and embedded in paraffin. Thin (4 μm) sections were deparaffinized and rehydrated. Slides were boiled in 10 mM citrate buffer for α-SMA and CD148. Sections were incubated with anti-a-SMA antibodies (Abcam, Cambridge, MA, no. ab5694) and for anti-CD148 antibodies (R&D systems, Minneapolis, MN, no. MAB1934) overnight at 4° C. Species-matched fluorescent-conjugated secondary antibodies were applied for staining at 37° C. for 1 h. Nuclei were stained with 4′,6-diamidino-2-phenylindole (DAPI). The slides were analyzed using an Olympus Inverted System Microscope IX70 (Olympus, Center Valley, PA), and photomicrographs were taken with a Nikon camera. CD148 fluorescence intensity was quantified by ImageJ. Mouse lungs were collected on Day 21, fixed with 4% (wt/vol) neutralized buffered paraformaldehyde, embedded in paraffin, and stained with hematoxylin and eosin (H+E) or Masson's trichrome.

### Western Immunoblot Analysis

Polyacrylamide gel electrophoresis and immunoblotting were performed according to standard methods. The electrophoresed proteins were transferred to polyvinylidene difluoride (PVDF) membranes by semidry electrophoretic transfer at 15 V for 60-75 min. The membranes were blocked overnight at 4° C. in 5% bovine serum albumin (BSA). The cells were incubated with primary antibodies diluted 1:500 in Tris-buffered saline/Tween 20 (TBS-T) containing 5% BSA for 2 h and then incubated with the secondary antibody at room temperature for 1 h. Suitable horseradish peroxidase (HRP)-conjugated secondary antibodies were used (1:5000 dilution in TBST containing 1% BSA). The signals were detected by ECL (Thermo Fisher Scientific, Waltham, MA). Quantification of protein bands was performed with the computer software ImageJ (Image Processing and Analysis in Java Edition: 1.29 URL: http://rsb.info.nih.gov/ij/NIH, Maryland) and was expressed as a ratio of band intensity with respect to the loading control.

### Quantitative Real-Time PCR (qPCR)

Total RNA was isolated with Trizol reagent (Invitrogen, Carlsbad, CA) and cDNA was synthesized from RNA (1 μg) using a SuperScript First-strand synthesis system for reverse transcription (RT) PCR (Invitrogen, Carlsbad, CA). Primer sequences are shown in the Table S1. RT-PCR, with SYBR Green Master Mix (Bio-Rad Laboratories, Hercules, CA, USA), was performed using the StepOnePlus Real-Time PCR System (Applied Biosystems, Foster City, CA, USA). The relative quantity of target mRNA was calculated by use of the CT method, or 2-AACT, and normalized by use of GAPDH as an endogenous control (Sequence Detection System software, version 1.7; Applied Biosystems).

### Lentiviral Transfection

For CD148 silencing experiments, the pLKO.1 plasmid, carrying the human shRNA PTPRJ/CD148 target sequence ACGAGTCGTCATCTAACTATA (consortium number TRCN0000320555) and pLKO.1, carrying a Scr sequence, were purchased from Sigma-Aldrich. For CD148 overexpression pLenti-GIII-CD148-HA (no. LV278210) and empty vector (EV, pLenti-GIII-HA) plasmids were purchased from (ABMgood, Vancouver, BC). Lentiviral particles were generated by use of a commercially available packaging mix, provided by MilliporeSigma (no. SHP001) or by ABMgood (no. LV003) in human embryonic kidney 293 T cells, according to the manufacturer's instructions. Lentiviral particle containing media was harvested and concentrated using a Centricon Plus-70 Centrifugal Filter (no. UFC700308, Millipore Sigma, St Louis, MO). Lung fibroblasts were infected with the lentiviral particles, and stably infected cells were selected by use of puromycin (10 μg/ml).

### Gel Contraction Assay

The assay was performed as previously described (Tsoyi K et al., Am J Respir Cell Mol Biol 2018; 58:208-215). Briefly, cell pellets of lung fibroblasts were mixed with 8 volumes of rat tail type I collagen suspension, one volume of 1× concentrated PBS and one volume of reconstitution buffer (2% sodium bicarbonate and 4.77% HEPES dissolved in 0.05 N NaOH) at a concentration of 2×10⁶ cells/ml. Cell-populated collagen solution was immediately poured into a 24-well-plate (0.5 ml/well) and incubated at 37° C. for 1 h to permit complete gelation. 16 h later, gels were gently transferred to 60 mm cell culture dish with a spatula and overlaid with culture media. Gel images were taken at 0, 12 and 24 h.

### Fas Ligand (Fasl) Treatment

Soluble recombinant human FasL was purchased from Enzo life sciences (Cat No: ALX-522-020) and dissolved in 1×PBS. Cells were treated with FasL at doses of 100 or 200 ng/ml and incubated for 24 hours. After incubation cell viability was measured using MTT assay or subjected to caspase-3 activity assay as described below.

### Cell Viability

Cell viability was determined using the 3-[4,5-dimethylthiazol-2-yl]-2,5-diphenyl tetrazolium bromide (MTT) assay and trypan blue exclusion assay using standard methods. Cells were seeded at 1×10⁴ cells/well in 24-well plates. After different treatments, 20 μl of 5 mg/ml MTT solution was added to each well (0.1 mg/well), and wells were incubated for 4 h. The supernatants were aspirated, the formazan crystals in each well were dissolved in 200 μL of dimethyl sulfoxide for 30 min at 37° C., and optical density at 570 nm was read on a microplate reader. For the trypan blue exclusion assay, cells were harvested and 10 μL of 0.4% trypan blue solution was added to 10 μL of cells collected from each well, and the cells were incubated for 2 min. Unstained live cells were counted on an automated cell counter.

### Caspase-3 Activity Assay

Caspase-3 activity in protein cell lysates was measured using a commercially available kit (no. K006-100, Biovision, San Francisco, CA).

### Luciferase Assay

3×10⁵ cells/well were plated in triplicate on 6-well plates and incubated for 24 h. Transient transfection assays in cells were performed by use of FuGENE 6 transfection reagent (Promega, Madison, WI). The construct carrying five copies of an NF-κB response element that drives transcription of the luciferase reporter gene (Promega, Madison, WI, no. 9PIE849, 500 ng/well) and a β-galactosidase expression plasmid (250 ng/well) to correct for transfection efficiency were co-transfected into mouse lung fibroblasts. The cells were harvested for luciferase activity using the Luciferase Assay System (Promega, Madison, WI), 6 h after treatment with TGF-β1 (10 ng/ml). Luciferase activity was measured in a Wallace Victor3 1420 multilabel counter (PerkinElmer, Waltham, MA). B-Galactosidase activity was measured using the mammalian-Galactosidase Assay Kit (Thermo Fisher Scientific, Waltham, MA).

### CD148 Gene Expression Analysis in Single Cell RNA Sequencing (scRNA-Seq) Dataset

From a published scRNA-seq dataset (Adams T S et al., Sci. Adv. 2020; 6(28): eaba1983), clusters labeled fibroblast and myofibroblast were extracted for analysis. Pseudo-samples were generated by summing all the counts matrices for a given patient sample within the cluster. A differential pseudo-bulk expression analysis comparing IPF over controls was performed using a negative binomial generalized linear model as implemented for EdgeR in the R-statistical programming environment.

### Statistical Analysis

Data are expressed as mean±SEM. Comparisons of mortality were made by analyzing Kaplan-Meier survival curves and log-rank tests to assess for differences in survival. For comparisons between two groups, we used Student's unpaired t test or Mann-Whitney's non-parametric test. Statistical significance was defined as P<0.05. One-way analysis of variance, followed by Newman-Keuls or Tukey's post-test analysis, or Kurskal-Wallis non-parametric test, was used for analysis of more than two groups. The numbers of samples per group (n), or the numbers of experiments, are specified in the figure legends.

### EXAMPLES

### CD148 is Downregulated in IPF

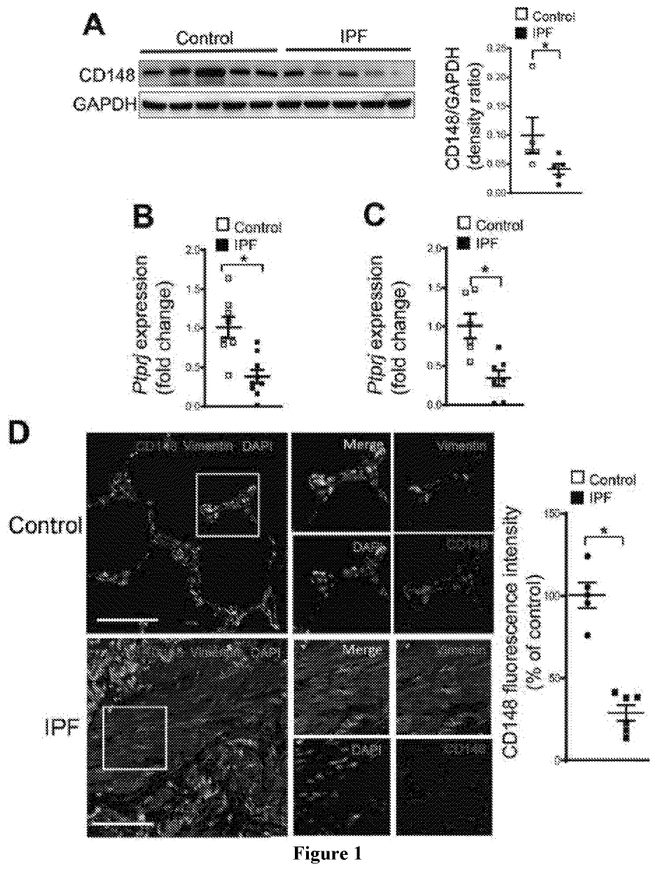

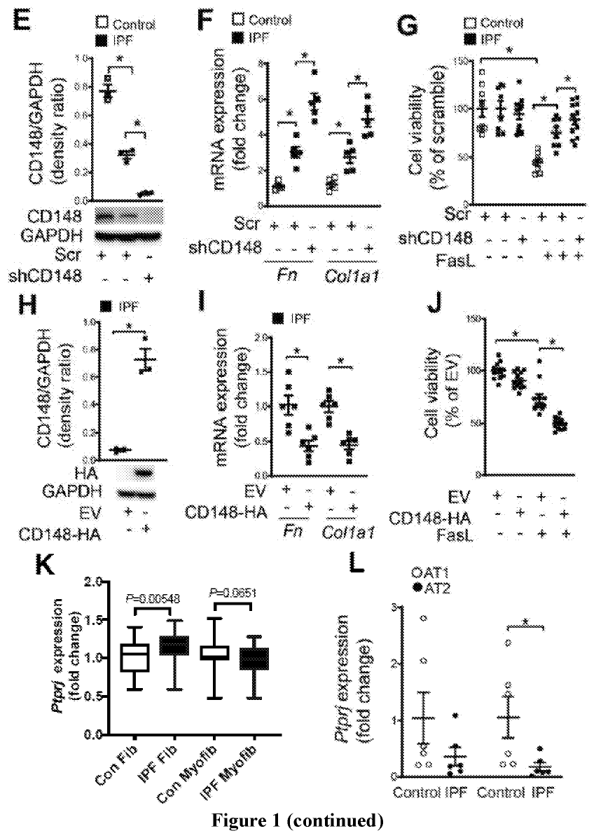

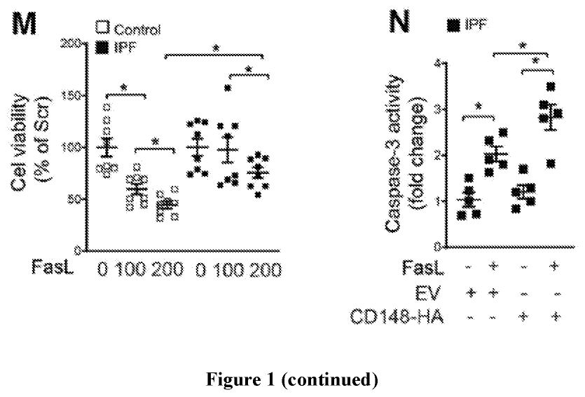

The expression of CD148 in IPF or control lungs and in cell isolates from these lungs was measured. CD148 protein and corresponding Ptprj mRNA levels were downregulated in IPF lungs compared to control lungs (FIG. 1, A-B). In IPF lung homogenates enriched for fibroblasts, Ptprj mRNA was downregulated relative to fibroblast-enriched control lung homogenates (FIG. 1C). Immunofluorescence staining revealed that CD148 co-stained with vimentin positive cells in control lungs, indicating its expression in fibroblasts, whereas CD148 staining was significantly reduced in IPF lungs (FIG. 1D). Analysis of a previously reported single cell RNA sequencing (scRNAseq) dataset for IPF lung revealed downregulated CD148 gene expression in IPF myofibroblasts compared to controls, which did not achieve statistical significance (FIG. 1K). CD148 gene expression was modestly increased in IPF fibroblasts compared to control fibroblasts (FIG. 1K). Finally, we determined that CD148 was also expressed in alveolar epithelial type I and type II (AT1 and AT2) cells from control lungs, with lower mRNA expression (FIG. 1L) in AT2 cells from IPF lungs.

### CD148 Regulates the Profibrotic Phenotype of Lung Fibroblasts Derived from IPF Patients

Fibroblasts isolated from IPF lungs have increased ECM production and resistance to cell death and apoptosis. To determine the role of CD148 in fibroblasts isolated from IPF lungs, we silenced CD148 expression in these cells using shCD148 (FIG. 1E). CD148 silencing resulted in higher gene expression of fibronectin (Fn) and collagen 1a1 (Col1a1) in IPF-derived lung fibroblasts relative to scramble (scr)-transfected IPF fibroblasts (FIG. 1F). Furthermore, IPF fibroblasts were resistant to cell death induced by Fas ligand (FasL) (FIG. 1M). This effect was enhanced in CD148-deficient cells (FIG. 1G). Overexpression of CD148 using stable lentiviral transduction with pLenti-CD148-HA (FIG. 1H) resulted in downregulation of ECM gene expression (Fn, Col1a1) (FIG. 1I), increased cell death (FIG. 1J) and enhanced caspase-3 activity (FIG. 1N).

### CD148 Deficiency in Fibroblasts Leads to Increased Pulmonary Fibrosis in Bleomycin (BLM)-Challenged Mice

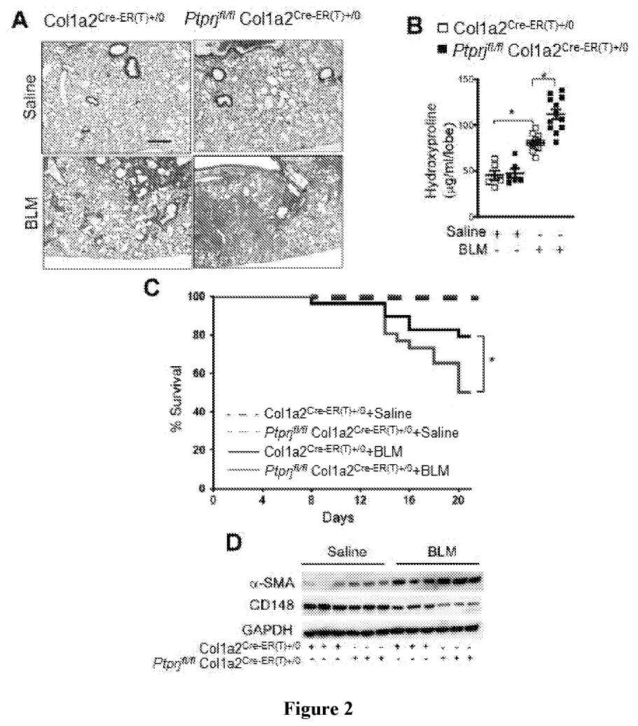

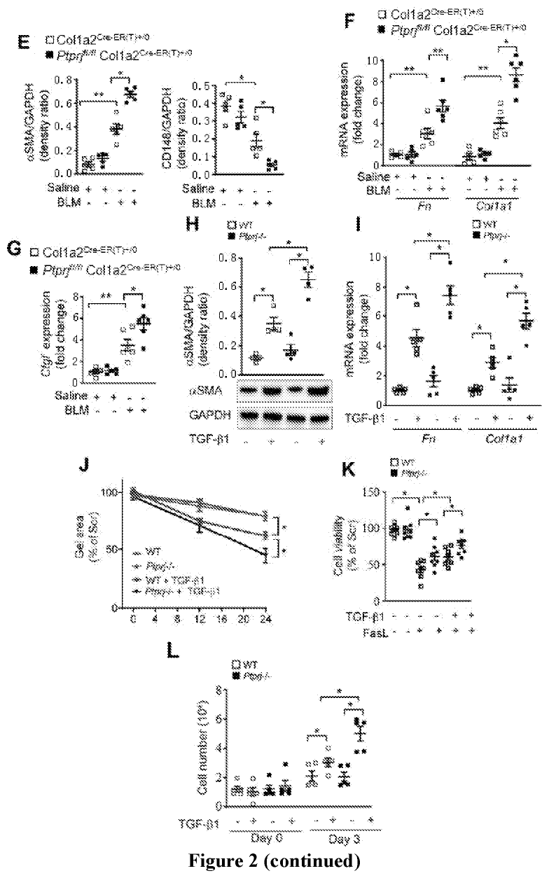

To investigate the role of CD148 in pulmonary fibrosis, we generated mice with a fibroblast-specific conditional deletion of CD148 (Ptprj^{fl/fl} Col1a2^{Cre-ER(T)+/0}). CD148 was deleted in lung fibroblasts isolated from tamoxifen-treated Ptprj^{fl/fl} Col1a2^{Cre-ER(T)+/0} mice. We exposed Ptprj^{fl/fl} Col1a2^{Cre-ER(T)+/0} mice or Col1a2^{Cre-ER(T)+/0} (control) mice to BLM to induce pulmonary fibrosis. At 21 days following BLM instillation, Ptprj^{fl/fl} Col1a2^{Cre-ER(T)+/0} mice displayed greater lung interstitial thickening compared to control mice. The fibroblast-specific CD148 deficient mice also displayed markedly higher lung collagen content, by Masson's Trichrome stain (FIG. 2A) and hydroxyproline measurements (111.8-18.5 vs. 80.8-8.8 μg/ml/lobe) (FIG. 2B), reduced survival (FIG. 2C), higher lung expression of a-SMA (FIG. 2, D-E) and profibrotic genes (Fn, Col1a1, and Ctgf) (FIG. 2, F-G) and reduced CD148 expression (FIG. 2, D-E). There was no difference in inflammatory cell counts in the bronchoalveolar lavage (BAL) fluid between the strains after BLM challenge.

### CD148 Deficiency Leads to Increased Myofibroblast Differentiation, ECM Production and Resistance to Apoptosis in Fibroblasts after TGF-β1 Stimulation

Lung fibroblasts from Ptprj^{fl/fl} Col1a2^{Cre-ER(T)+/0} and Col1a2^{Cre-ER(T)+/0} (control) mice were isolated. CD148 deficiency enhanced TGF-β1-induced myofibroblast differentiation, as determined by a-SMA expression (FIG. 2H) and increased the ECMgene expression (Col1a1 and Fn) (FIG. 2I). Cell contractility was higher in Ptprj−/− fibroblasts compared to wild type cells after TGF-β1 treatment (FIG. 2J). TGF-β1 stimulation induced resistance to FasL-induced cell death and apoptosis in wild-type cells, which was exacerbated in Ptprj−/− fibroblasts (FIG. 2K). CD148 deficiency also increased cell proliferation induced by TGF-β1 in fibroblasts (FIG. 2L).

### CD148 Deficiency Upregulates TGF-β1-Induced PI3K/Akt/mTOR Signaling

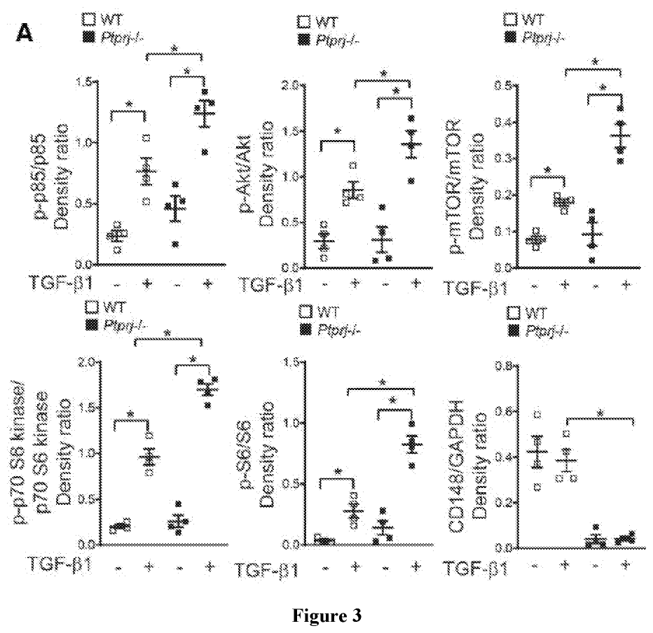

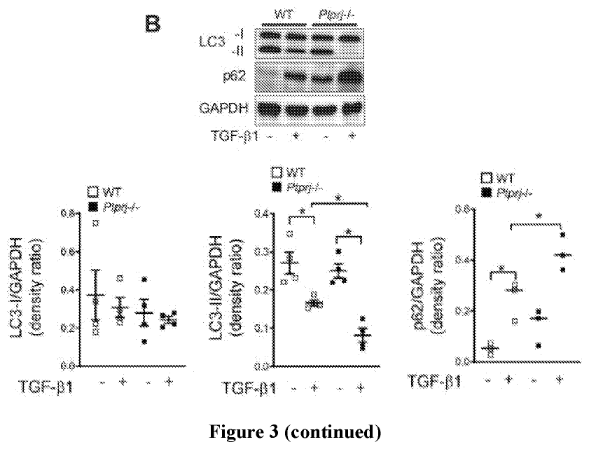

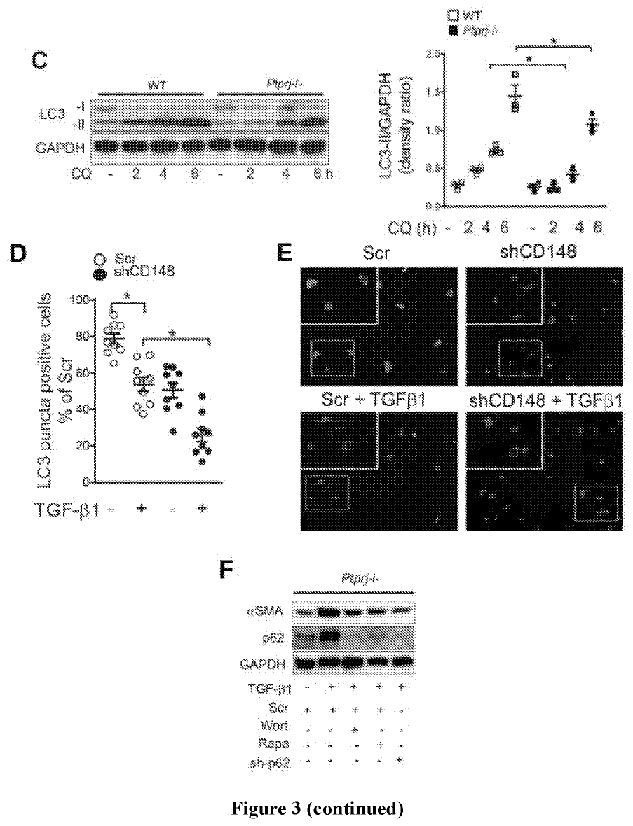

CD148 regulates PI3K signaling by dephosphorylating (inactivating) the regulatory subunit of PI3K (p85). As PI3K/Akt signaling is upregulated in activated fibroblasts and contributes to the development of pulmonary fibrosis, we investigated whether CD148 deficiency can enhance PI3K/Akt/mTOR signaling induced by TGF-β1 in lung fibroblasts derived from Ptprj^{fl/fl} Col1a2^{Cre-ER(T)+/0} mice. TGF-β1 treatment upregulated the phosphorylation of PI3K (p85 subunit), Akt, mTOR and mTOR-related signaling proteins (p70 S6-kinase and S6) (FIG. 3A). CD148 deficiency further increased the expression of phospho (p)-PI3K, p-Akt, p-mTOR, and downstream targets, p-p70 and p-S6 (FIG. 3A).

### CD148 Deficiency Inhibits Autophagy and Leads to p62 Accumulation in Lung Fibroblasts

Activation of mTOR suppresses autophagy in IPF-derived fibroblasts (Romero Y et al., Aging Cell 2016; 15:1103-1112). As the absence of CD148 leads to activation of mTOR, a regulator of autophagy, we hypothesized that CD148 deficiency could potentially modulate autophagy. TGF-β1 downregulated autophagy in lung fibroblasts, as reflected by reduced expression of microtubule associated protein-1 light chain-3B (LC3)-II (active form) relative to LC3-I, and increased p62 expression (FIG. 3B). CD148 deficiency exacerbated the suppression of autophagy by TGF-β1 (FIG. 3B). We also examined relative autophagic flux using LC3 turnover assay. Wild type and Ptprj−/− fibroblasts were treated with chloroquine which inhibits autophagosome-lysosome fusion and consumption of LC3B; this causes an accumulation of LC3-II that reflects the rate of autophagosome formation in the presence or absence of TGF-β1. CD148 deficient fibroblasts exhibited delayed LC3-II accumulation compared to control fibroblasts (FIG. 3C). Decreased autophagosome formation was also observed in LC3-GFP transgenic lung fibroblasts in the absence of CD148 (FIG. 3, D-E). We next investigated whether the PI3K/mTOR axis and p62 upregulation are essential for the pro-fibrotic response in CD148 deficient cells. We found that increase of a-SMA in CD148-deficient cells in response to TGF-β1 was attenuated by wortmannin (PI3K inhibitor), rapamycin (mTOR inhibitor) and by p62 knockdown using sh-p62 (FIG. 3F).

### p62-Dependent NF-κB Activation Drives Transcriptional Regulation of Profibrotic Gene Expression

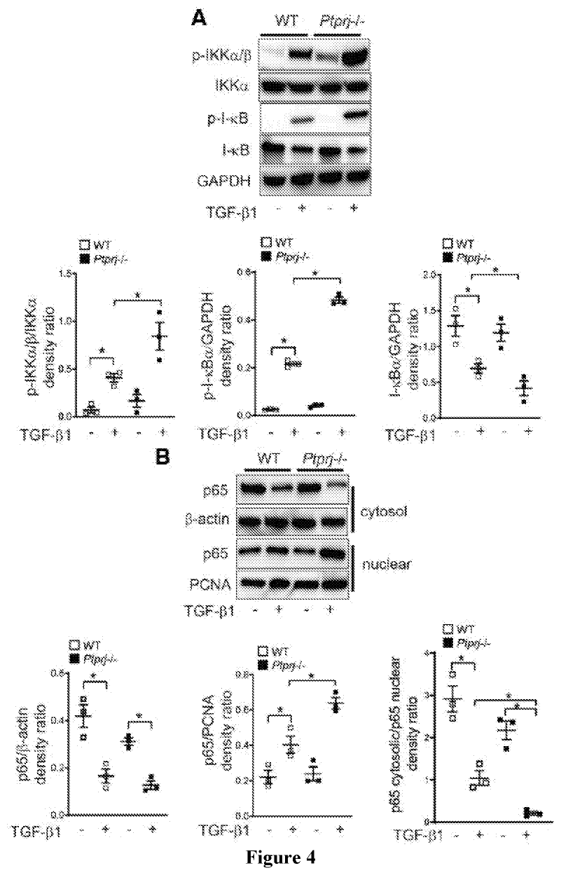

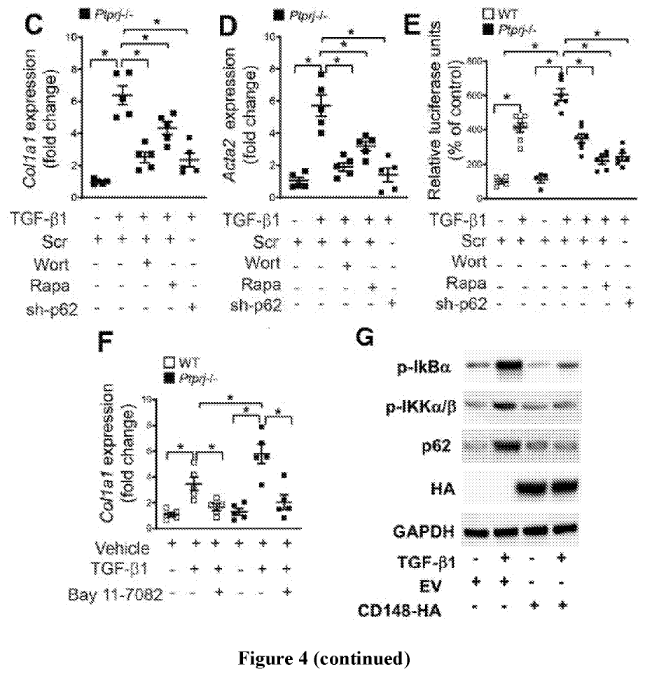

In cancer cells, p62 accumulation activates NF-κB by phosphorylating the inhibitor of kappa-B kinase (IKK), resulting in degradation of the kappa-B inhibitor (I-κB) and NF-κB nuclear translocation. We thus sought to determine the relevance of this pathway in fibrogenesis. CD148-deficient cells (Ptprj−/−) had higher levels of p-IKKα/β and p-I-κB (FIG. 4A), and higher nuclear accumulation of NF-κB (p65 subunit) (FIG. 4B) in response to TGF-β1. In CD148-deficient lung fibroblasts, Col1a1 and Acta2 mRNA expression levels were dependent on PI3K/Akt, mTOR and p62 levels in response to TGF-β1 (FIG. 4, C-D). Interestingly, NF-κB activity in CD148-deficient cells was also dependent on PI3K/Akt, mTOR and increased p62 levels (FIG. 4E). The NF-κB inhibitor Bay 11-7082 inhibited TGF-β1-dependent increase in Col1a1 expression in CD148-deficient cells (FIG. 4F). Taken together, we demonstrate for the first time that decreased autophagy may transcriptionally modulate profibrotic gene expression by enhancing a p62/NF-κB signaling axis. Furthermore, we demonstrate that CD148 counteracts this signaling axis by inhibiting the PI3K/Akt/mTOR pathway. We also further confirmed the role of CD148 in regulating this pathway by overexpressing CD148 in human lung fibroblasts. As shown in FIG. 4G, overexpression of CD148 attenuated p62, p-IKKα/β, p-I-κB expression and inhibited NF-κB luciferase activity in response to TGF-β1.

### An 18-Aa SDC2-ED Derived Peptide (SDC2-Pep, Also Named QM107) Inhibits Pulmonary Fibrosis Via CD148 In Vivo and In Vitro

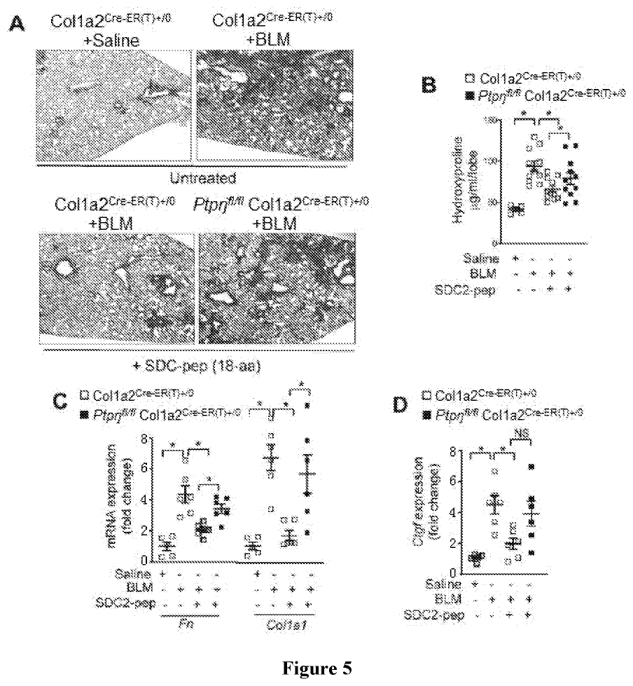

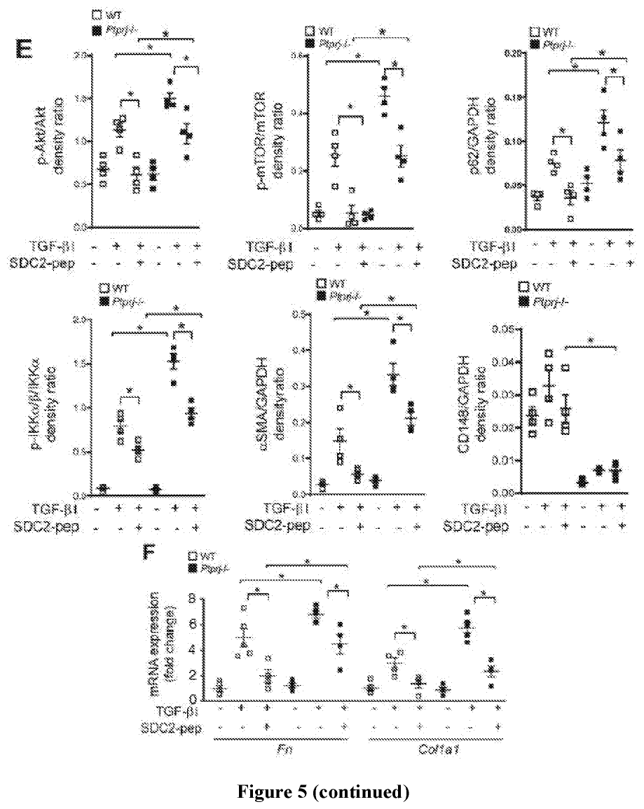

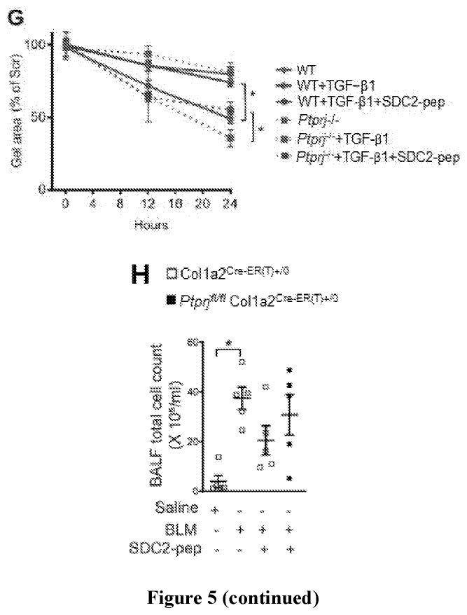

Two known extracellular proteins, thrombospondin and syndecan-2 (SDC2), can bind to CD148 and activate PTP activity (Whiteford J R et al., Mol Biol Cell 2011; 22:3609-3624; Takahashi K et al., Proc Natl Acad Sci USA 2012; 109:1985-1990). Furthermore, we have previously identified an 18-aa sequence of the SDC2 ectodomain responsible for binding and activation of CD148 in endothelial cells (De Rossi G et al., J Cell Sci 2014; 127:4788-4799). We therefore evaluated the therapeutic potential of a SDC2-peptide (SDC2-pep) in the mouse model of BLM-induced fibrosis. We exposed Ptprj^{fl/fl} Col1a2^{Cre-ER(T)+/0} mice or Col1a2^{Cre-ER(T)+/0} (control) mice to BLM followed by administration of SDC2-pep beginning on day 10 after BLM, once daily, for 5 consecutive days. The SDC2-pep significantly inhibited pulmonary fibrosis in control mice, while in Ptprj^{fl/fl} Col1a2^{Cre-ER(T)+/0} mice the antifibrotic effect was significantly reduced but not absent (FIG. 5, A-C), suggesting CD148-independent effects in lung fibroblasts or other cells in the fibrotic niche. SDC2-pep inhibited profibrotic gene expression (Fn, Col1A1, Ctgf) in control mice (FIG. 5, C-D). There was a trend towards decreased BAL total cell counts in control mice treated with SDC2-pep but not in Ptprj^{fl/fl} Col1a2^{Cre-ER(T)+/0} mice (FIG. 5H) after BLM.

Next, fibroblasts were stimulated with TGF-β1 in the absence or presence of SDC2-pep. TGF-β1 treatment increased the expression of p-Akt, p-mTOR, p62, p-IKKα/β, and α-SMA in wild type fibroblasts (FIG. 5E). This effect was markedly abrogated by treatment with SDC2-pep. In contrast, the inhibitory effect of SDC2-pep on TGF-β1-dependent expression of these signaling proteins in Ptprj−/− fibroblasts was reduced (FIG. 5E). Similarly, SDC2-pep inhibited TGF-β1-induced Fn and Col1a1 gene expression (FIG. 5F) and cell contractility (FIG. 5G) in wild type fibroblasts. SDC-2 partially inhibited these effects in Ptprj−/− fibroblasts, which had markedly enhanced pro-fibrotic responses to TGF-β1 stimulation (FIG. 2, G-I).

### SDC2-Pep Inhibits Pulmonary Fibrosis in Human IPF Fibroblasts and Ex Vivo Precision Cut Lung Slices (PCLS)

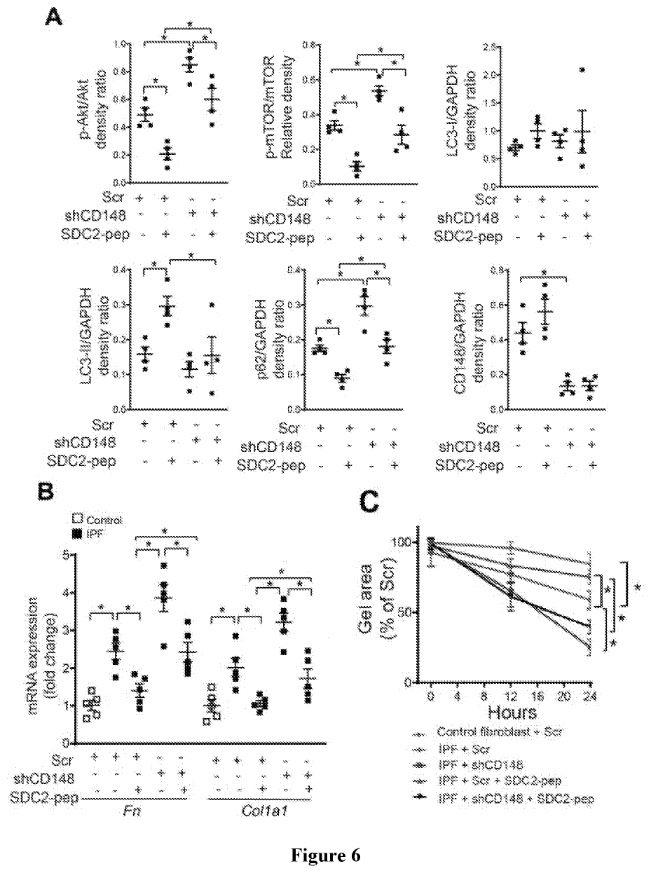

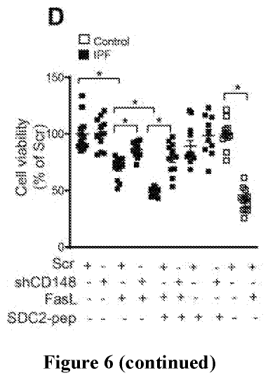

We next evaluated the therapeutic potential of SDC2-pep in human IPF fibroblasts. Treatment with SDC2-pep significantly inhibited the activation of the PI3K/Akt/mTOR pathway in human IPF fibroblasts (FIG. 6A). SDC2-pep significantly inhibited the expression of p-Akt and p-mTOR in human IPF fibroblasts in scr-transfected but not in shCD148-transfected cells. SDC2-pep restored autophagy in human IPF fibroblasts in a CD148-dependent manner, as evidenced by increased relative LC3B-II expression and reduced p62 expression. SDC2-pep also inhibited ECM gene expression (Fn, Col1a1) (FIG. 6A) and cell contractility (FIG. 6B) and these effects were reduced in the absence of CD148. Furthermore, SDC2-pep significantly enhanced FasL-induced cell death in IPF fibroblasts and to a lesser extent in CD148-deficient cells (FIG. 6D).

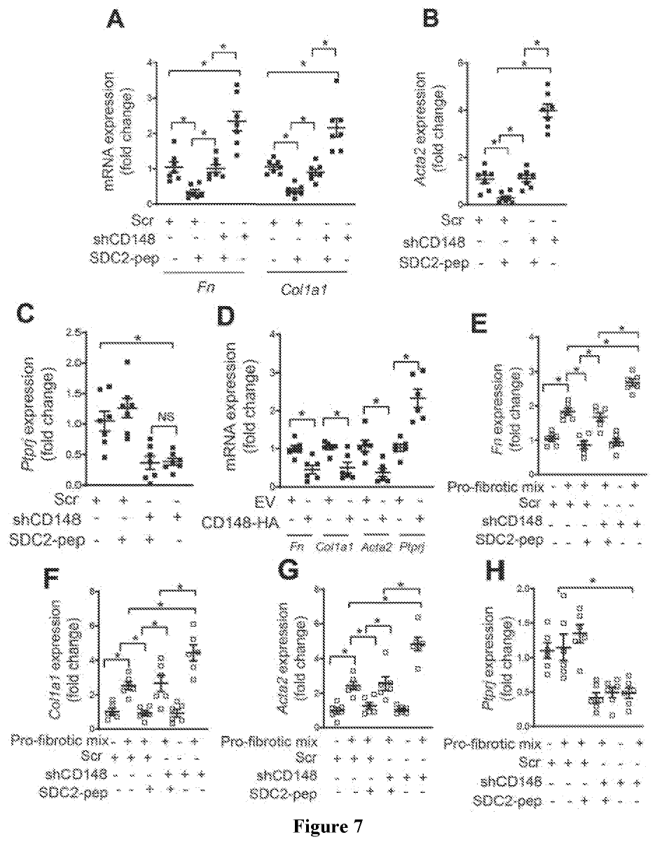

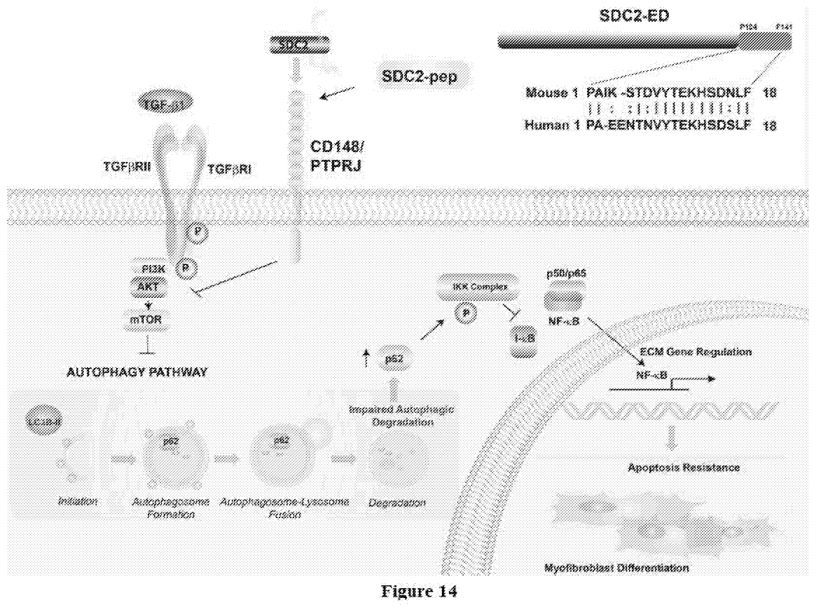

Finally, we tested the potential anti-fibrotic effect of SDC2-pep in precision cut lung slices (PCLS) derived from IPF lung explants obtained at the time of transplant. We exposed PCLS from IPF patients to SDC2-pep, which resulted in significantly decreased profibrotic gene expression (Col1a1, Fn, Acta2) in scr-transfected PCLS and this effect was reduced in shCD148-transfected PCLS (FIG. 7A-B). Flow cytometry in both fibroblasts and epithelial cells isolated from PCLS indicated that shCD148 reduced gene expression by approximately 60%. SDC2-pep did not affect Ptprj gene expression in PCLS (FIG. 7C). Whereas shCD148 transfection of PCLS resulted in increased pro-fibrotic responses (FIG. 7A, B). Conversely, lentiviral overexpression of CD148 in PCLS from IPF lungs inhibited profibrotic gene expression (Col1a1, Fn, Acta2) (FIG. 7D). Finally, PCLS from control lungs were stimulated with a profibrotic mix in the presence or absence of SDC2-pep. SDC2-pep attenuated ECM gene expression (Col1a1, Fn, Acta2) but not Ptprj expression in response to stimulation with a profibrotic mix in control PCLS. SDC-2 pep also partially inhibited ECM expression in CD148 deficient PCLS, which had markedly increased pro-fibrotic responses to profibrotic mix (FIG. 7, E-H). Taken together, our findings demonstrate that SDC2-pep inhibits lung fibrosis in human and experimental models of IPF predominantly through CD148, although CD148-independent effects may also contribute to its observed therapeutic effects. Our findings represent a new paradigm for the role of CD148 as a therapeutic target in IPF (FIG. 14).

### QM107 Inhibits TGF-1 Induced αSMA Production in Mouse Lung Fibroblasts

Mouse lung fibroblasts (Mlg2009 cells) were treated with TGF-β1 (10 ng/ml) with or without QM107 (at the doses indicated). After 24 hours cells were lysed and analysed by western blot for αSMA expression. The expression of αSMA in response to TGF-β1 is indicative of the differentiation of the fibroblasts to myofibroblasts, which is an important process during fibrosis. P<0.05; significant comparisons: * vs. WT, † vs. WT+TGFβ1. As shown in FIG. 9, QM107 inhibited TGF-β1 induced αSMA production in mouse lung fibroblasts in a dose-dependent manner.

### QM107 Inhibits TGF-β1 Stimulated Matrix Production in Mouse Lung Fibroblasts

The expression of pro-fibrotic genes (collagen1a1 and fibronectin) was measured in mouse lung fibroblasts (Mlg2009 cells) treated with TGF-β1 (10 ng/ml) and the indicated doses of QM107. Gene expression was measured by qRTPCR. P<0.05; significant comparisons: * vs. WT, † vs. WT+TGFβ1. As shown in FIG. 10, QM107 significantly inhibited TGF-β1 stimulated matrix production in mouse lung fibroblasts.

### Topical Application of QM107 in the Bleomycin Lung Fibrosis Model Leads to a Reduction in Collagen Deposition

QM107 attenuates Bleomycin induced lung fibrosis. WT mice (n=12 each group) were intratracheally injected with saline or bleomycin (0.75 mg/kg). After 14 days, QM107 (0.5 mg/kg) was delivered intranasally 4 times every second day. On day 24 lungs were harvested and assayed for hydroxyproline content, which is a measure of collagen production. The results are shown in FIG. 11, P<0.05; significant comparisons: * vs. con.

### Histochemical Staining of Bleomycin Treated Lungs Reveals QM107 Leads to Less Collagen Deposition and Less Structural Damage

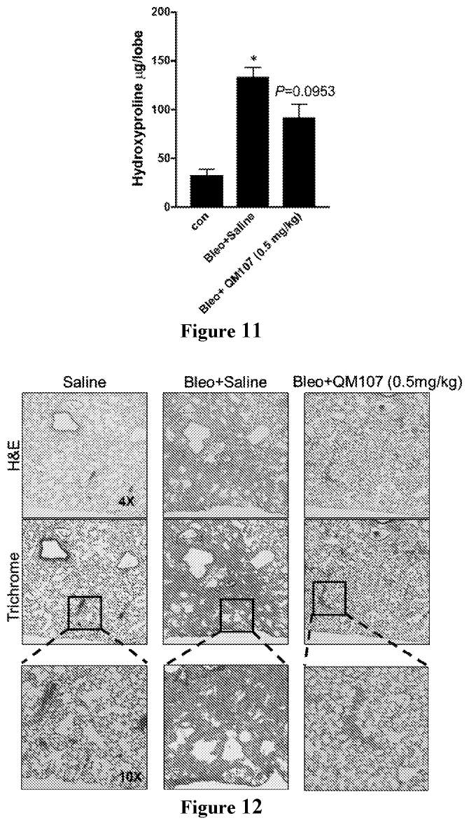

Mice were treated with control, bleomycin or bleomycin plus QM107. The lung sections from each group were collected and subjected to H and E and Trichrome staining. Collagen staining (Blue) is greatly decreased in QM107 treated lungs, and the lung architecture is less disrupted (FIG. 12, scale bar=2 mm).

### QM 107 Attenuates Pro-Fibrotic Gene Expression in Precision Cut Lung Slices from IPF Patients

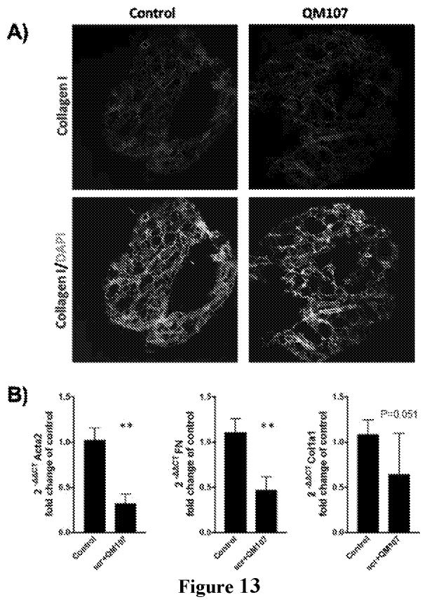

Precision cut lung slices (100u) were cultured for 72 hours in the presence of either QM107 (0.5 μM) or PBS (Control) prior to immuno-staining for collagen I (red) and DAPI (blue). Collagen deposition was notably reduced in QM107 treated slices (FIG. 13A).

Pro-fibrotic gene expression was also greatly reduced in QM107 lung slices (FIG. 13B). This was true of α-smooth muscle actin (Acta2), Fibronectin (FN) and Collagen I (Col1a1). P<0.001; significant comparisons: ** vs. control.

1. An anti-fibrotic peptide comprising or consisting of an amino acid sequence having at least 66%, 70%, 72%, 75%, 77%, 80%, 83%, 85%, 88%, 90%, 94%, 95% or 100% identity to the sequence of QM107 or mQM107.

2. The anti-fibrotic peptide according to claim 1, wherein the peptide consists of up to 20, 25, 30, 40, 50, 60, 70, 80, 90 or 100 amino acids and comprises an amino acid sequence having at least 66%, 70%, 72%, 75%, 77%, 80%, 83%, 85%, 88%, 90%, 94%, 95% or 100% identity to the sequence of QM107 or mQM107.

3. The anti-fibrotic peptide according to claim 1, wherein the peptide comprises or consists of an amino acid sequence having at least 66%, 70%, 72%, 75%, 77%, 80%, 83%, 85%, 88%, 90%, 94%, 95% or 100% identity to up to 20, 25, 30, 40, 50, 60, 70, 80, 90 or 100 consecutive amino acid residues derived from human syndecan-2 or mouse syndecan-2.

4. The anti-fibrotic peptide according to claim 1, wherein the peptide consists of up to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180 or 190 amino acid residues.

5. The anti-fibrotic peptide according to claim 1 wherein the peptide is fused to a heterologous peptide.

6. The anti-fibrotic peptide according to claim 1 wherein the peptide is conjugated to a carrier molecule.

7. A source of the anti-fibrotic peptide according to claim 1.

8. The source according to claim 7, wherein the source is a polynucleotide which expresses the anti-fibrotic peptide, a polypeptide which is enzymatically cleavable to produce the anti-fibrotic peptide, a cell which expresses the anti-fibrotic peptide, or a composition which releases the anti-fibrotic peptide.

9. A pharmaceutical composition comprising the anti-fibrotic peptide according to or a source of the anti-fibrotic peptide according to claim 1, and a pharmaceutically acceptable carrier.

10. A method of inhibiting αSMA expression in cells, comprising exposing the cells to the anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier.

11. The method of claim 10, wherein the αSMA expression is induced by TGF-β1.

12. The method of claim 10, wherein the cells are lung cells, particularly fibroblasts.

13. A method of inhibiting matrix production in cells, comprising exposing the cells to the anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier.

14. The method of claim 13, wherein matrix production is induced by TGF-β1.

15. The method of claim 13, wherein the cells are lung cells, particularly fibroblasts.

16. A method of inhibiting pro-fibrotic protein expression in cells, comprising exposing the cells to the anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier, preferably the pro-fibrotic protein includes at least one or two selected from the group consisting of collagen 1a1 and fibronectin.

17. The method of claim 16, wherein pro-fibrotic protein expression is induced by TGF-β1.

18. The method of claim 16, wherein the cells are lung cells, particularly fibroblasts.

19. A method of treating fibrosis, a fibrotic condition or a fibrotic symptom in a subject in need thereof, comprising administering to the subject a therapeutically effective amount of the anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier.

20. The method of claim 19, wherein the fibrosis, fibrotic condition or fibrotic symptom is induced by TGF-β1, bleomycin or by radiation.

21. The method of claim 19, wherein the subject has pulmonary fibrosis.

22. The method of claim 19, wherein the administration is topical administration, particularly intranasal administration.

23. The method of claim 19, wherein the anti-fibrotic peptide, the source or the pharmaceutical composition is in a spray composition or inhalation composition.

24. A method of inhibiting collagen deposition in the lung of a subject in need thereof, comprising administering to the subject a therapeutically effective amount of the anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier.

25. A method of inhibiting structural damage in the lung of a subject in need thereof, comprising administering to the subject a therapeutically effective amount of the anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier.

26. The method of claim 19, wherein the subject has a fibrotic condition selected from the group consisting of glycogen storage disease type III (GSD III), glycogen storage disease type VI (GSD VI), glycogen storage disease type IX (GSD IX), nonalcoholic steatohepatitis (NASH), cirrhosis, hepatitis, scleroderma, alcoholic fatty liver disease, atherosclerosis, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, pulmonary fibrosis, idiopathic pulmonary fibrosis, diffuse parenchymal lung disease, idiopathic interstitial fibrosis, diffuse interstitial fibrosis, interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-poly myositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis, or symptoms or sequelae thereof, or other diseases or conditions resulting in the excessive deposition of extracellular matrix components, such as collagen, which may be affected by interventions within the TRJ3 pathway, dust lung, sarcoidosis, lung fibrosis induced by drug or radiation and fibrogenic alveolitis, and a combination thereof.

27. The method of claim 10, wherein the subject is suffering a disease selected from the group consisting of idiopathic pulmonary fibrosis (IPF), hypersensitivity pneumonitis, dust lung, sarcoidosis, lung fibrosis induced by drug or radiation and fibrogenic alveolitis.

28. A method of treating a cancer in a subject in need thereof, comprising administering radiation therapy in combination with a therapeutically effective amount of the anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier.

29. A method of treating a cancer in a subject who receives radiation therapy, comprising administering radiation therapy in combination with a therapeutically effective amount of the anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier.

30. A method of treating a cancer in a subject in need thereof or in a subject who receives radiation therapy comprising administering radiation therapy in combination with a therapeutically effective amount of the anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier, wherein the anti-fibrotic peptide, the source, or the pharmaceutical composition is administered before, simultaneously or after radiation therapy.

31. The anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier, for use in a method of inhibiting αSMA expression in cells, comprising exposing the cells to the anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier.

32. Use of the anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier for the manufacture of a medicament for a method of inhibiting αSMA expression in cells, comprising exposing the cells to the anti-fibrotic peptide according to claim 1, a source of the anti-fibrotic peptide according to claim 1, or a pharmaceutical composition comprising the anti-fibrotic peptide according to claim 1 or a source of the anti-fibrotic peptide according to claim 1, and a pharmaceutically acceptable carrier.
